(19) United States
(12) Patent Application Publication
(10) Pub. No.: US 2021/0386652 A1
(43) Pub. Date: Dec. 16, 2021

(54) OIL-IN-WATER EMULSION COSMETIC

(71) Applicant: Shiseido Company, Ltd., Chuo-ku, Tokyo (JP)

(72) Inventors: Yuko NAGARE, Tokyo (JP); Mayuri TASHIRO, Tokyo (JP)

(73) Assignee: Shiseido Company, Ltd., Chuo-ku, Tokyo (JP)

(21) Appl. No.: 17/284,273

(22) PCT Filed: Oct. 10, 2019
(86) PCT No.: PCT/JP2019/039979

§ 371 Date: Apr. 9, 2021

(30) Foreign Application Priority Data
Oct. 12, 2018 (JP) 2018-193008

(51) Int. Cl. A61K 8/87; A61K 8/06; A61K 8/29; A61K 8/891; A61K 8/58; A61Q 1/02; A61K 8/27

(52) U.S. Cl. CPC A61K 8/87; A61K 8/062; A61K 8/29; A61K 2800/43; A61K 8/585; A61Q 1/02; A61K 8/27; A61K 8/891

(57) ABSTRACT

The purpose of the present invention is to provide an oil-in-water emulsion cosmetic that is thickened using an associative thickener containing a hydrophobic modified polyether urethane, that stably holds a hydrophobization powder in an inner oil phase, and that does not release water even after long-term storage, without a reduction in usage sensations regarding moistness, resilience, and smoothness originally possessed by the oil-in-water emulsion cosmetic. The present invention is an oil-in-water emulsion cosmetic characterized by comprising (A) a hydrophobic modified polyether urethane having a specific structure such as polyurethane-59, (B) a polyether modified silicone having an HLB (Si) of 5-14, and (C) a hydrophobization powder. The cosmetic according to the present invention preferably further comprises (D) a lower alcohol and/or a polyol, and/or (E) a nonionic surfactant having an HLB of 8-18. The hydrophobization powder (C) is preferably a metal oxide powder treated with triethoxycaprylylsilane, dimethicone, hydrogen dimethicone, or a metallic soap.

### TECHNICAL FIELD

The present invention relates to an oil-in-water emulsion cosmetic. More specifically, the present invention relates to an oil-in-water emulsion cosmetic in which a hydrophobically modified polyether urethane having a specific structure is blended and which stably holds a hydrophobized powder in the inner oil phase while having a resilient sensation on use, and causes no water release.

### BACKGROUND ART

In the technical field of pharmaceutical products, cosmetic products, and the like, various thickeners are blended to hold the dosage form and stability thereof or to improve sensation on use. In oil-in-water emulsion cosmetics that provide moist and fresh sensations upon applying on the skin, water-soluble thickeners are widely used. Above all, an associative thickener comprising a hydrophobically modified polyether urethane is known as a water-soluble thickener that is excellent in not only viscosity stability but also texture.

Patent Document 1 describes that an associative thickener comprising a hydrophobically modified polyether urethane is combined with a thickener comprising the microgel obtained by radical polymerization in the dispersion phase in which water-soluble ethylenically unsaturated monomers are dispersed in the dispersion phase, in a predetermined amount blended, so that not only a synergistically increased thickening effect can be obtained but also a new sensation excellent in resilience can be obtained.

However, the emulsion cosmetic in which a (PEG-240/decyltetradeceth-20/HDI) copolymer as the hydrophobically modified polyether urethane described in Patent Document 1 is blended may cause stickiness, and the addition of the powder of silicic anhydride and the like can suppress the stickiness but causes water release, so that the amount of the powder that can be blended with the (PEG-240/decyltetradeceth-20/HDI) copolymer has been limited (Patent Document 2).

On the other hand, BB (Blemish Balm) cream has been attracting attention as a cosmetic having a skin correction effect and a skin protection (ultraviolet protection effect) effect, and an attempt to prepare an oil-in-water BB cream has also been made (Patent Document 3). However, blending the (PEG-240/decyltetradeceth-20/HDI) copolymer which is an associative thickener into the oil-in-water emulsion cosmetic containing a powder caused problems that the thickening effect cannot be sufficiently exerted and the sensation on use and the stability are also reduced.

In Patent Document 4, the aforementioned problems are solved by further blending a polysaccharide thickener and a specific dispersing agent, but the conventional system thickened with the (PEG-240/decyltetradeceth-20/HDI) copolymer has a low static viscosity and sometimes causes separation of water (water release) after long-term storage.

### CITATION LIST

### Patent Literature

### Patent Document 1: JP-A 2007-291026

### Patent Document 2: JP-A 2014-040385

### Patent Document 3: JP-A 2013-193999

### Patent Document 4: JP-A 2009-234917

### SUMMARY OF INVENTION

### Technical Problem

The present invention has been made in view of the aforementioned shortcomings of the conventional techniques and intends to provide an oil-in-water emulsion cosmetic that stably holds the hydrophobized powder in the inner oil phase and causes no separation of water (water release) even after long-term storage without impairing the fresh, resilient, and smooth sensation on use originally possessed by the oil-in-water emulsion cosmetic thickened with the associative thickener comprising the hydrophobically modified polyether urethane.

### Solution to Problem

The present inventors have conducted intensive studies to solve the aforementioned problems and as a result, found that blending a hydrophobically modified polyether urethane having a specific structure as an associative thickener and combining with a polyether modified silicone having a moderate (5 to 14) HLB (Si) enables to prevent water release (separation of water) even by blending a significant amount of hydrophobized powder and to maintain a unique sensation on use, thereby completed the present invention.

That is, the present invention provides an oil-in-water emulsion cosmetic comprising:

(A) a hydrophobically modified polyether urethane having a specific structure;

(B) a polyether modified silicone having an HLB (Si) of 5 to 14; and

(C) a hydrophobized powder.

### Advantageous Effects of Invention

The oil-in-water emulsion cosmetic of the present invention not only exerts shape recovery ability and unique and resilient sensation on use possessed by the hydrophobically modified polyether urethane, but also causes no separation of water (water release) observed in conventional hydrophobically modified polyether urethane-blended cosmetics and is stable.

### DESCRIPTION OF EMBODIMENTS

The oil-in-water emulsion cosmetic of the present invention contains (A) a hydrophobically modified polyether urethane having a specific structure, (B) a polyether modified silicone having an HLB (Si) of 5 to 14, and (C) a hydrophobized powder as essential components. Hereinafter, the present invention will be described in detail.

(A) Hydrophobically Modified Polyether Urethane Having a Specific Structure

The hydrophobically modified polyether urethane having a specific structure (component A) used in the present invention is a urethane-based copolymer (also referred to as associative thickener) having a hydrophilic group portion as a skeleton and a hydrophobic portion at an end thereof, and it is considered that the hydrophobic portions of the copolymer are associated with one another in an aqueous medium and the hydrophilic portion thereof forms a loop and/or a bridge to exert a thickening action.

The hydrophobically modified polyether urethane having a specific structure in the present invention is a urethane-based polymer obtained by reacting:

a monohydroxy compound (I) represented by the following general formula (1):

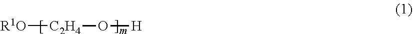

wherein R¹ is an aliphatic hydrocarbon group having 24 to 36 carbon atoms, and m represents a number of 0 to 1,000;

a polyethylene glycol (II) represented by the following general formula (2):

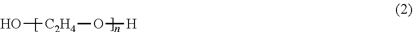

wherein n represents a number of 2 to 1,000;

a monoglyceryl ether compound (III) represented by the following general formula (3):

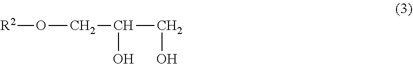

wherein R² represents an aliphatic hydrocarbon group having 5 to 12 carbon atoms; and

an isocyanate compound (IV) represented by the following general formula (4):

R³ [symbol] NCO]_{q} (4)

wherein R³ represents a hydrocarbon group having 4 to 13 carbon atoms, and q represents a number of 2 or 3.

The urethane-based polymer used in the present invention is obtained by reacting the above compounds (I) to (IV). Specifically, the hydroxyl groups contained in each of the compounds (I), (II), and (III) are reacted with the isocyanate group contained in the compound (IV). Since there are three types of compounds having the hydroxyl group and two types of them are divalent, the obtained polymer has a complex structure, so that it cannot be represented by an appropriate general formula.

The production method of the urethane-based polymer used in the present invention is not particularly specified as long as four compounds are reacted, and each of the compounds may be reacted at once or may be separately reacted. However, since the reaction does not occur even if any of the compounds (I) to (III) is charged into the reaction system after the isocyanate compound (IV) has been completely reacted, it is preferable to mix the compounds (I) to (III) in advance and to add the isocyanate compound (IV) thereto to cause the reaction. Specifically, the compounds (I) to (III) are charged into the reaction system and melted and mixed at 40 to 100° C., preferably at 60 to 80° C., and the isocyanate compound (IV) is added into the reaction system to react while maintaining the same temperature. Thereafter, the reaction system may be aged at the same temperature for 30 minutes to 3 hours until the reaction is completed.

In the above reaction, the blending ratio of each component is not particularly specified, but it is preferable that the monohydroxy compound (I) be 10 to 30 mol, the monoglyceryl ether compound (III) be 5 to 20 mol, and the isocyanate compound (IV) be 20 to 50 mol relative to 10 mol of polyethylene glycol (II), and it is more preferable that the monohydroxy compound (I) be 15 to 25 mol, the monoglyceryl ether compound (III) be 8 to 15 mol, and the isocyanate compound (IV) be 25 to 40 mol relative to 10 mol of polyethylene glycol (II), because the function as a viscosity modifier is favorable and the reaction can be easily controlled.

The detail of the urethane-based polymer is described in Japanese Patent No. 6159738.

As the hydrophobically modified polyether urethane having a specific structure (component A) that is an essential component in the present invention, a urethane-based polymer in which the monohydroxy compound (I) is polyethylene glycol ether of tetradecyloctadecanol (tetradecyloctadeceth-100), the polyethylene glycol (II) is PEG-240, the monoglyceryl ether compound (III) is ethylhexylglycerin, and the isocyanate compound (IV) is hexamethylene diisocyanate is particularly preferable.

The copolymer of tetradecyloctadeceth-100, PEG-240, ethylhexylglycerin, and hexamethylene diisocyanate (HDI) is referred to as “polyurethane-59” in International Nomenclature of Cosmetic Ingredients (INCI name). Polyurethane-59 is particularly preferably used as the hydrophobically modified polyether urethane having a specific structure (component A) in the present invention.

A commercial product may be used as polyurethane-59, and for example, “ADEKANOL GT-930” manufactured by ADEKA is preferably used. This product is provided as a mixture containing polyurethane-59 (about 30% by mass), butylene glycol (about 55% by mass), tocopherol (about 0.05% by mass), and water (about 14.95% by mass) (hereinafter also referred to as “composition containing polyurethane-59”).

The amount of the (A) hydrophobically modified polyether urethane having a specific structure (for example, polyurethane-59) blended in the cosmetic of the present invention is 0.1 to 5% by mass, and preferably 0.2 to 3% by mass of the composition, in the actual amount thereof.

In addition to the component A (for example, polyurethane-59), the cosmetic of the present invention may contain another hydrophobically modified polyether urethane that does not have the aforementioned specific structure. Preferred examples of another hydrophobically modified polyether urethane include (PEG-240/decyltetradeceth-20/HDI) copolymer which has conventionally been widely used. Examples of the commercial product thereof include “ADEKANOL GT-700” or “ADEKANOL GT-730” manufactured by ADEKA.

In the cosmetic of the present invention, the proportion of the hydrophobically modified polyether urethane having a specific structure (for example, polyurethane-59) to the total amount of the hydrophobically modified polyether urethane to be blended is not particularly limited, but is 30% by mass or more, preferably 40% by mass or more, and more preferably 50% by mass or more based on the total amount of the hydrophobically modified polyether urethane, from the viewpoint of ensuring the suppression of the separation of water.

(B) Polyether Modified Silicone Having HLB (Si) of 5 to 14

The polyether modified silicone having an HLB (Si) of 5 to 14 (component B) used in the present invention is a silicone derivative having a polyoxyalkylene group selected from polyoxyethylene (POE) and polyoxypropylene (POP). In particular, the polyether modified silicone represented by the following general formula is preferable.

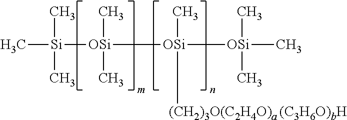

In the above formula, m is 1 to 1,000, preferably 5 to 500, and n is 1 to 40. It is preferable that m:n be 200:1 to 1:1. Further, a is 5 to 50, and b is 0 to 50.

The molecular weight of polyether modified silicone is not particularly limited, but is suitably 3,000 to 60,000, and particularly suitably in a range of 3,000 to 40,000. With the use of the polyether modified silicone having a low molecular weight, a particularly excellent texture can be achieved.

The polyether modified silicone used in the present invention is selected from those having an HLB (Si) of 5 to 14, and preferably those having an HLB (Si) of 7 to 14. The HLB (Si) as used herein is a value obtained by the following calculation expression.

$$\frac{\begin{matrix}( Molecular weight of polyoxyethlene ( POE ) \\ and polyoxyethylene ( POE ) )\end{matrix}}{Molecular weight } \times 20$$

As the polyether modified silicone having an HLB (Si) of 5 to 14 (component B), one or two or more selected from those conventionally used in the cosmetic and the like can be used. Specific examples thereof include PEG/PPG-19/19 dimethicone, PEG/PPG-30/10 dimethicone, PEG-9 dimethicone, PEG-12 dimethicone, and PEG-11 methyl ether dimethicone.

The polyether modified silicone having an HLB (Si) of 5 to 14 (component B) used in the present invention may be a commercial product and examples thereof include, but are not limited to, the following.
Trade name BY11-030 (manufactured by Dow Corning Toray Co., Ltd.: PEG/PPG-19/19 dimethicone, HLB (Si)=7.7)
Trade name SH3773M (manufactured by Dow Corning Toray Co., Ltd.: PEG-12 dimethicone, HLB (Si)=7.7)
Trade name KF6013 (manufactured by Shin-Etsu Chemical Co., Ltd.: PEG-9 dimethicone, HLB (Si)=10)
Trade name BY25-339 (manufactured by Dow Corning Toray Co., Ltd.: PEG/PPG-30/10 dimethicone, HLB (Si)=12.2)
Trade name KF6011 (manufactured by Shin-Etsu Chemical Co., Ltd.: PEG-11 methyl ether dimethicone, HLB (Si)=12.7)
Trade name SH3771M (manufactured by Dow Corning Toray Co., Ltd.: PEG-12 dimethicone, HLB (Si)=13)

In the cosmetic of the present invention, the amount of the polyether modified silicone having an HLB (Si) of 5 to 14 (component B) blended is preferably 0.1% by mass or more, 0.2% by mass or more, 0.3% by mass or more, 0.4% by mass or more, or 0.5% by mass or more, and preferably 10% by mass or less, 5% by mass or less, or 3% by mass or less relative to the total amount of the cosmetic. The specific range of the amount blended is 0.1 to 10% by mass, preferably 0.5 to 5% by mass, and more preferably 0.5 to 3% by mass. When the amount blended is less than 0.1% by mass, the following hydrophobized powder (component C) is not uniformly dispersed, so that the emulsion particle size may be large and the cosmetic may be unstable, and when it is more than 10% by mass, the cosmetic may be sticky.

(C) Hydrophobized Powder

The hydrophobized powder (component C) in the oil-in-water emulsion cosmetic of the present invention is powder particles that use powder particles of metal oxides and the like as the base material, to the surface of which the hydrophobic treatment is applied.

The base material of the hydrophobized powder is not particularly limited as long as it is a powder component used in the cosmetic, and examples thereof include metal oxides, for example, titanium oxide, iron oxide, magnesium oxide, zinc oxide, calcium oxide, and aluminum oxide. Also, composite powder particles comprising a plurality of materials can be used as the base material.

The shape and the size of the powder particles such as metal oxides which may be used as the base material are not particularly limited and the shape includes fine particles (average particle size=about 1 μm or less) and pigment grades. As the shape, for example, shapes such as spherical, plate-like, petal-like, flake-like, rod-like, spindle, needle, and irregular shapes may be adopted.

The hydrophobic treatment agent applied to these base material powder particles is not particularly limited as long as they are used in the cosmetic and the like, and examples thereof include dimethicone, hydrogen dimethicone, alkylsilane, amino acid, lipoamino acid, metallic soap, lecithin, and alginic acid. In particular, a silicone-based treatment agent or a metallic soap is preferably selected, and among them, a silicone-based treatment agent is preferable from the viewpoint of stable dispersibility. Examples of the metallic soap include aluminum stearate and aluminum dimyristate. Examples of the silicone-based treatment agent include a silicone oil such as hydrogen dimethicone, methylhydrogenpolysiloxane, dimethylpolysiloxane (dimethicone), and methylphenylpolysiloxane; alkylsilane such as methyltriethoxysilane, ethyltriethoxysilane, hexyltriethoxysilane, and triethoxycaprylylsilane; and fluoroalkyl silane such as trifluoromethylethyltrimethoxysilane and heptadecafluorodecyltrimethoxysilane. Among them, triethoxycaprylylsilane, dimethicone, and hydrogen dimethicone are particularly preferable.

In the oil-in-water emulsion cosmetic of the present invention, the amount of the hydrophobized powder (component C) blended is 0.5% by mass or more, 1% by mass or more, 2% by mass or more, 3% by mass or more, 4% by mass or more, or 5% by mass or more, and 35% by mass or less, 30% by mass or less, 25% by mass or less, or 20% by mass or less relative to the total amount of the cosmetic. The specific range of the amount blended is 0.5 to 35% by mass, preferably 3 to 25% by mass, and more preferably 5 to 20% by mass. When the amount blended is less than 0.5% by mass, the effect of containing the powder cannot be sufficiently exerted, and when it is more than 35% by mass, there is a tendency of causing problems in texture such as friction feeling, crinkles, and stickiness.

The oil constituting the inner phase of the oil-in-water emulsion cosmetic of the present invention is not particularly limited and may include oils that are liquid or solid or semisolid at ambient temperature and pressure (nonvolatile oils) and volatile oils.

The nonvolatile oil can be conveniently defined as an oil having a boiling point higher than about 250° C., and includes a hydrocarbon oil, an ester oil, a plant oil, a higher alcohol, a higher fatty acid, an oily ultraviolet absorber, and a silicone oil.

Examples of the hydrocarbon oil include liquid paraffin, paraffin, squalane, squalene, pristane, and vaseline.

Examples of the ester oil include isopropyl myristate, cetyl octanoate, octyldodecyl myristate, isopropyl palmitate, butyl stearate, hexyl laurate, myristyl myristate, decyl oleate, hexyldecyl dimethyloctanoate, cetyl lactate, myristyl lactate, acetylated lanolin, isocetyl stearate, isocetyl isostearate, cholesteryl 12-hydroxystearate, ethylene glycol di 2-ethylhexanoate, dipentaerythritol fatty acid ester, N-alkyl glycol monoisostearate, neopentyl glycol dicaprate, diisostearyl malate, glycerol di 2-heptyl undecanoate, trimethylolpropane tri 2-ethylhexanoate, trimethylolpropane triisostearate, glycerol trioctanoate, glycerol triisopalmitate, cetyl 2-ethylhexanoate, pentaerythritol tetra 2-ethylhexanoate, glycerol tri 2-ethylhexanoate, 2-ethylhexyl palmitate, glycerol trimyristate, glyceride tri 2-heptylundecanoate, castor oil fatty acid methyl ester, oleyl oleate, acetoglyceride, 2-heptylundecyl palmitate, diisobutyl adipate, N-lauroyl-L-glutamic acid-2-octyldodecyl ester, di-2-heptylundecyl adipate, ethyl laurate, di-2-ethylhexyl sebacate, 2-hexyldecyl myristate, 2-hexyldecyl palmitate, di 2-hexyldecyl adipate, diisopropyl sebacate, and triethyl citrate.

Examples of the plant oil include avocado oil, camellia oil, macadamia nut oil, corn oil, olive oil, rapeseed oil, sesame oil, castor oil, peanut oil, almond oil, soybean oil, tea seed oil, jojoba oil, and germ oil.

Examples of the higher alcohol include oleyl alcohol, isostearyl alcohol, octyldodecanol, decyl tetradecanol, jojoba alcohol, cetyl alcohol, and myristyl alcohol. Examples of the higher fatty acid include oleic acid, isostearic acid, linoleic acid, linolenic acid, eicosapentaenoic acid, docosahexaenoic acid, palmitic acid, and stearic acid.

The oily ultraviolet absorber is not particularly limited as long as it is usually used in cosmetic products. For example, ultraviolet absorbers selected from the group consisting of octocrylene, octylmethoxycinnamate, 4-tert-butyl-4′-methoxydibenzoylmethane, methylene bis benzotriazolyl tetramethylbutylphenol, bis ethylhexyloxyphenol methoxyphenyl triazine, diethylamino hydroxybenzoyl hexyl benzoate, ethylhexyl triazone, diethylhexylbutamidotriazone, 2-hydroxy 4-methoxybenzophenone, benzalmalonate, and benzotriazole may be appropriately combined and used.

As a volatile hydrocarbon oil, a hydrocarbon oil having a relatively low molecular weight (the boiling point is about 250° C. or less) can be used, and specific examples thereof include light liquid isoparaffin, isododecane, and isohexadecane.

The silicone oil is selected from volatile and nonvolatile linear, branched, or cyclic silicone oils. Examples thereof include methylpolysiloxane, methylphenylpolysiloxane, methylpolycyclosiloxane, methylhydrogenpolysiloxane, dimethylsiloxane, dimethylsiloxane methyl (POE) siloxane copolymer, highly polymerized methylpolysiloxane, dimethylsiloxane methyl (POP) siloxane copolymer, tetradecamethylhexasiloxane, octamethyltrisiloxane, dimethylsiloxane methylcetyloxysiloxane copolymer, decamethyltetrasiloxane, cyclopentasiloxane, octamethylcyclotetrasiloxane, decamethylcyclopentasiloxane, dodecamethylcyclohexasiloxane, and hexadecamethylcycloheptasiloxane.

In the oil-in-water emulsion cosmetic of the present invention, the amount of the oil blended is not particularly limited, but is typically 3 to 25% by mass, and preferably 5 to 20% by mass relative to the total amount of the cosmetic.

In the cosmetic of the present invention, it is preferable to blend the ester oil in the oil, and the amount of the ester oil blended is preferably about 30% by mass or more, and more preferably about 50% by mass or more, relative to the amount of the hydrophobized powder (component C) blended.

The aqueous components constituting the outer phase of the oil-in-water emulsion cosmetic of the present invention contain water and water-soluble components. In particular, it is preferable to blend a lower alcohol (a monohydric alcohol having 5 or less carbon atoms) such as ethanol and/or a polyol (component D) such as 1,3-butylene glycol, dipropylene glycol, and glycerin in terms of the stability of the system. The amount of the lower alcohol and the polyol blended is preferably about 0.1 to 20% by mass relative to the total amount of the cosmetic.

In the cosmetic of the present invention, the stability can be further improved by blending a nonionic surfactant (component E) having an HLB of 8 to 18, and preferably an HLB of 10 to 18. Noted that the “HLB (Hydrophilic-Lypophilic Balance)” as used herein is an index indicating the balance between the hydrophilicity and lipophilicity of the nonionic surfactant and defined to be HLB value=7+11.7 log (sum of formula weights of hydrophilic moieties/sum of formula weights of lipophilic moieties) by the Kawakami method.

The nonionic surfactant (component E) having an HLB of 8 to 18 is not particularly limited, but an ethylene oxide addition type nonionic surfactant is particularly preferable, and examples thereof include POE (10 to 50 mol) phytosterol ether, POE (10 to 50 mol) dihydrocholesterol ether, POE (10 to 50 mol) 2-octyldodecyl ether, POE (10 to 50 mol) decyltetradecyl ether, POE (10 to 50 mol) oleyl ether, POE (10 to 50 mol) cetyl ether, POE (5 to 30 mol) POP (5 to 30 mol) 2-decyltetradecyl ether, POE (10 to 50 mol) POP (2 to 30 mol) cetyl ether, POE (20 to 60 mol) sorbitan monooleate, POE (10 to 60 mol) sorbitan monoisostearate, POE (10 to 80 mol) glyceryl monoisostearate, POE (10 to 30 mol) glyceryl monostearate, and POE (20 to 100) hydrogenated castor oil derivative. Noted that the “POE” refers to polyoxyethylene and the “POP” refers to polyoxypropylene. Further specific examples thereof include PEG-25 hydrogenated castor oil, PEG-30 hydrogenated castor oil isostearate, PEG-40 hydrogenated castor oil, PEG-60 hydrogenated castor oil, and PEG-40 hydrogenated castor oil laurate; polyoxyethylene fatty acid glyceryl and polyoxyethylene fatty acid sorbitan.

In addition to the aforementioned components, other optional components that may be blended into oil-in-water emulsion cosmetics, in particular, BB cream and the like can be blended into the oil-in-water emulsion cosmetic of the present invention in a range not impairing the effects of the present invention.

Examples of other optional components include, but are not limited to, a hydrophilic thickener, a lipophilic thickener, a moisturizing agent, a water-soluble ultraviolet absorber such as a benzylidene camphor derivative (for example, terephthalylidene camphor sulfonic acid) and a phenylbenzimidazole derivative, a pH adjuster, a neutralizing agent, an antioxidant, a preservative, a chelating agent, an emollient, a plant extract, a fragrance, a pigment, and various agents.

The oil-in-water emulsion cosmetic of the present invention can be produced according to a conventional method. For example, it can be produced by separately mixing aqueous phase components and oil phase components, adding the hydrophobized powder to the mixture of oil phase components and subjecting the mixture to a dispersion treatment using a homomixer and the like, and then adding the oil phase mixture in which the powder is dispersed to the aqueous phase mixture and emulsifying the mixture using a homomixer and the like.

The cosmetic of the present invention has a smaller emulsion particle size and is more stable as compared to the conventional emulsion which combines a surfactant having a low HLB (less than 5) and a volatile oil. The emulsion particle size of the inner oil phase in the cosmetic of the present invention is not particularly limited, but is preferably 0.5 to 5 μm. The emulsion particle size is preferable as small as possible, in terms of stability, and for example, it is further preferably 4 μm or less, 3.5 μm or less, or 3 μm or less. With the emulsion particle size exceeding 5 μm, tendency of lowering of dispersion stability is observed.

In the cosmetic of the present invention, the hydrophobic powder is stably dispersed in the inner oil phase and no separation of water is observed even it is allowed to stand for a long period of time.

### EXAMPLES

Hereinafter, the present invention will be described in more detail by way of specific examples, but the present invention is not limited to the following examples. Unless otherwise specified, the amount blended in the following Examples and the like indicates % by mass.

Oil-in-water emulsion cosmetics having compositions listed in the following Tables 1 to 3 were prepared. Specifically, they were prepared by uniformly mixing each of the aqueous phase part and the oil phase part, dispersing the powder part in the oil phase part using a homomixer, adding this mixture to the aqueous phase part, and then emulsifying the mixture using a homomixer.

The cosmetic obtained in each example was evaluated with respect to the shape recovery ability, the separation of water, and the aggregation of the powder, according to the following criteria.

<Evaluation Criteria>

### (1) Shape Recovery Ability

After the surface of the cosmetic of each example that was filled up to 90% of a cream jar container made of plastic (50 ml capacity) was scooped once with a laboratory spatula, the container was allowed to stand at ordinary temperature for 1 hour and the appearance at that time point was visually observed.

A: The surface was flat after 1 hour.

D: The surface was not flat after 1 hour.

(2) Separation of Water

The cosmetic of each example that was filled up to 90% of a cream jar container made of plastic (50 ml capacity) was stored at ordinary temperature for 3 months and the surface at that time point was visually observed and the presence or absence of the separation of water was confirmed.

Yes: Separation of water was observed.

No: No separation of water was observed.

(3) Aggregation of Powder

The cosmetic of each example that was filled up to 90% of a cream jar container made of plastic (50 ml capacity) was observed and determined visually and with an optical microscope (400× magnification).

A: No aggregation of the powder was observed neither visually nor with a microscope.

B: No aggregation of the powder was observed visually, but some aggregation of the powder was observed with a microscope.

C: The aggregation of the powder was observed both visually and with a microscope.

D: The powder forms large aggregates and the composition was segregated.

TABLE 1

| | | | Comparative | Comparative |
|---|---|---|---|---|
| | Example 1 | Example 2 | Example 1 | Example 2 |
| Ion-exchange water | Balance | Balance | Balance | Balance |
| PEG-40 hydrogenated castor oil | 1.0 | 1.0 | 1.0 | 1.5 |
| Glycerin | 2 | 2 | 2 | 2 |
| Succinoglycan | 0.2 | 0.2 | 0.2 | 0.2 |
| Composition containing polyurethane- | 2 | 2 | 2 | — |
| 59 *1 | | | | |
| (PEG-240/decyltetradeceth-20/HDI) | 0.5 | 0.5 | 0.5 | 1 |
| copolymer *2 | | | | |
| Chelating agent | 0.05 | 0.05 | 0.05 | 0.05 |
| Buffer | q.s. | q.s. | q.s. | q.s. |
| Triethoxycaprylylsilane-treated | 5 | 5 | 5 | — |
| pigment grade titanium oxide | | | | |
| Triethoxycaprylylsilane-treated iron | 0.2 | 0.2 | 0.2 | — |
| oxide (red) | | | | |
| Triethoxycaprylylsilane-treated iron | 0.5 | 0.5 | 0.5 | — |
| oxide (yellow) | | | | |
| Triethoxycaprylylsilane-treated iron | 0.01 | 0.01 | 0.01 | — |
| oxide (black) | | | | |
| Tetrahydro | — | — | — | 5 |
| tetramethylcyclotetrasiloxane/tetradecene- | | | | |
| treated pigment grade titanium oxide | | | | |
| Tetrahydro | — | — | — | 0.2 |
| tetramethylcyclotetrasiloxane/tetradecene- | | | | |
| treated iron oxide (red) | | | | |
| Tetrahydro | — | — | — | 0.5 |
| tetramethylcyclotetrasiloxane/tetradecene- | | | | |
| treated iron oxide (yellow) | | | | |
| Tetrahydro | — | — | — | 0.01 |
| tetramethylcyclotetrasiloxane/tetradecene- | | | | |
| treated iron oxide (black) | | | | |
| 1,3-Butylene glycol | 3 | 3 | 3 | 3 |
| Ethyl alcohol | 5 | 5 | 5 | 5 |
| PEG12-dimethicone (HLB (Si) = 7.7) *3 | 2 | — | — | — |
| PEG12-dimethicone (HLB (Si) = 13) *4 | — | 2 | — | — |
| PEG10-dimethicone (HLB (Si) = 4.5) *5 | — | — | 2 | 0.5 |
| Isopropyl myristate | 2 | 2 | 2 | — |
| Polypropylene glycol | 1 | 1 | 1 | — |
| Ethylhexyl methoxy cinnamate | 5 | 5 | 5 | 5 |
| Cyclopentasiloxane | — | — | — | 20 |
| Methylphenylpolysiloxane | — | — | — | 3 |
| Cetyl 2-ethylhexanoate | — | — | — | 3 |
| Isostearic acid | — | — | — | 0.5 |
| otal | 100 | 100 | 100 | 100 |
| Shape recovery ability | A | A | — | A |
| Separation (room temperature, stored | No | No | — | Yes |
| for 3 months) | | | | |
| Aggregation of powder | A | A | x | B |
| Emulsion particle size | 3 μm or | 3 μm or | Emulsion | 3-10 μm |
| | less | less | destruction | |

*1 ADEKANOL GT930 (manufactured by ADEKA)
*2 ADEKANOL GT700 (manufactured by ADEKA)
*3 SH3773M (manufactured by Dow Corning Toray Co., Ltd.)
*4 SH3771M (manufactured by Dow Corning Toray Co., Ltd.)
*5 KF-6017P (manufactured by Shin-Etsu Chemical Co., Ltd.)

TABLE 2

| | Example 3 | Example 4 | Example 5 |
|---|---|---|---|
| Ion-exchange water | Balance | Balance | Balance |
| PEG-60 hydrogenated castor oil | 0.5 | 0.5 | 0.5 |
| Glycerin | 2 | 2 | 2 |
| Succinoglycan | 0.2 | 0.2 | 0.2 |
| Composition containing polyurethane-59 *1 | 2 | 2 | 2 |
| (PEG-240/decyltetradeceth-20/HDI) copolymer *2 | 0.5 | 0.5 | 0.5 |
| Chelating agent | 0.05 | 0.05 | 0.05 |
| Buffer | q.s. | q.s. | q.s. |
| Tetrahydro | — | — | — |
| tetramethylcyclotetrasiloxane/tetradecene-treated | | | |
| pigment grade titanium oxide | | | |
| Tetrahydro | — | — | — |
| tetramethylcyclotetrasiloxane/tetradecene-treated | | | |
| iron oxide (red) | | | |
| Tetrahydro | — | — | — |
| tetramethylcyclotetrasiloxane/tetradecene-treated | | | |
| iron oxide (yellow) | | | |
| Tetrahydro | — | — | — |
| tetramethylcyclotetrasiloxane/tetradecene-treated | | | |
| iron oxide (black) | | | |
| Dimethicone-treated silica-coated fine particle zinc | 10 | — | — |
| oxide | | | |
| Hydrogen dimethicone-treated fine particle titanium | — | 10 | — |
| oxide | | | |
| Aluminum stearate-treated pigment grade titanium | — | — | 10 |
| oxide | | | |
| 1,3-Butylene glycol | 3 | 3 | 3 |
| Ethyl alcohol | 6 | 6 | 6 |
| PEG12-dimethicone (HLB (Si) = 7.7) *3 | 1.5 | 1.5 | 1.5 |
| Isopropyl myristate | 2 | 2 | 2 |
| Ethylhexyl methoxy cinnamate | 7.5 | 7.5 | 7.5 |
| Ethylhexyl triazone | 3 | 3 | 3 |
| otal | 100 | 100 | 100 |
| Shape recovery ability | A | A | A |
| Separation (room temperature, stored for 3 months) | No | No | No |
| Aggregation of powder | A | A | B |
| Emulsion particle size (μm) | 3 μm or | 3 μm or | 5 μm or |
| | less | less | less |

TABLE 3

| | | | Comparative | Comparative |
|---|---|---|---|---|
| | Example 6 | Example 7 | Example 3 | Example 4 |
| Ion-exchange water | Balance | Balance | Balance | Balance |
| PEG-40 hydrogenated castor oil | 0.5 | 0.5 | 0.5 | 0.5 |
| Glycerin | 2 | 2 | 2 | 2 |
| Succinoglycan | 0.2 | 0.2 | 0.2 | 0.2 |
| Composition containing polyurethane- | 3.5 | 2 | — | — |
| 59 *1 | | | | |
| (PEG-240/decyltetradeceth-20/HDI) | — | 0.5 | 0.8 | — |
| copolymer *2 | | | | |
| Composition containing alkyl | — | — | — | 4 |
| acrylates/steareth-20 methacrylate | | | | |
| copolymer *6 | | | | |
| Chelating agent | 0.05 | 0.05 | 0.05 | 0.05 |
| Buffer | q.s. | q.s. | q.s. | q.s. |
| Triethoxycaprylylsilane-treated | 5 | 5 | 5 | 5 |
| pigment grade titanium oxide | | | | |
| Triethoxycaprylylsilane-treated iron | 0.2 | 0.2 | 0.2 | 0.2 |
| oxide (red) | | | | |
| Triethoxycaprylylsilane-treated iron | 0.5 | 0.5 | 0.5 | 0.5 |
| oxide (yellow) | | | | |
| Triethoxycaprylylsilane-treated iron | 0.01 | 0.01 | 0.01 | 0.01 |
| oxide (black) | | | | |
| 1,3-Butylene glycol | 3 | 12 | 3 | 3 |
| Ethyl alcohol | 6 | | 6 | 6 |
| PEG12-dimethicone (HLB (Si) = 7.7) *3 | 1.5 | 1.5 | 1.5 | 1.5 |
| Isopropyl myristate | 2 | 2 | 2 | 2 |
| Ethylhexyl methoxy cinnamate | 7.5 | 7.5 | 7.5 | 7.5 |
| Ethylhexyl triazone | 3 | 3 | 3 | 3 |
| Total | 100 | 100 | 100 | 100 |
| Shape recovery ability | A | A | A | D |
| Separation (room temperature, stored | No | No | Yes | Yes |
| for 3 months) | | | | |
| Aggregation of powder | A | A | A | D |
| Emulsion particle size (μm) | 3 μm or | 3 μm or | 3 μm or | 3-5 μm |
| | less | less | less | |

*6 Aculyn 22 (manufactured by Rohm and Haas Company)

As apparent from the results in Table 1, Examples 1 and 2 in which polyurethane-59 was blended as the (A) hydrophobically modified polyether urethane having a specific structure and the (B) polyether modified silicone having an HLB (Si) of 5 to 14 was used, were excellent in shape recovery ability, had no aggregation of the powder, and caused no separation of water. However, when the polyether modified silicone having a low HLB (Si) (4.5) was used, the hydrophobized powder was not favorably dispersed (Comparative Example 1). On the other hand, in the conventional cosmetic that contains only (PEG-240/decyltetradeceth-20/HDI) copolymer as the hydrophobically modified polyether urethane (Comparative Example 2), the aggregation of the powder could be suppressed to some extent even with the use of the polyether modified silicone having a low HLB (Si) and the cosmetic also had shape recovery ability. However, it could not eliminate the problem of the separation of water.

As seen from the results in Table 2, in the cosmetic in which polyurethane-59 as the (A) hydrophobically modified polyether urethane having a specific structure and the (B) polyether modified silicone having an HLB (Si) of 5 to 14 were blended, favorable results were obtained even when the surface treatment agent of the hydrophobized powder was changed from triethoxycaprylylsilane (Examples 1 and 2) to other silicone-based treatment agents (hydrogen dimethicone and dimethicone) or a metallic soap (aluminum stearate) (Examples 3 to 5). In particular, when the silicone-based surface treatment agents were used, aggregation of the powder was completely suppressed.

As apparent from the results in Table 3, when only polyurethane-59 as the hydrophobically modified polyether urethane having a specific structure was blended, or another hydrophobically modified polyether urethane ((PEG-240/decyltetradeceth-20/HDI) copolymer) was blended in addition to polyurethane-59, they were excellent in both suppression of separation of water and powder dispersibility (suppression of aggregation). On the other hand, the separation of water could not be suppressed in Comparative Example 3 in which no polyurethane-59 was contained and only (PEG-240/decyltetradeceth-20/HDI) copolymer was blended. Further, it was found that Comparative Example 4 in which only another urethane-based thickener was blended caused separation of water and had no shape recovery ability.

Formulation examples other than the cosmetic of the present invention will be illustrated below. However, the present invention is not limited to these examples.

### Formulation Example 1: Sunscreen Cream

| | amount |
|---|---|
| Blended components | blended |
| Ion-exchange water | balance |
| Ethyl alcohol | 5 |
| Glycerin | 4 |
| PEG-60 hydrogenated castor oil | 1.5 |
| Xanthan gum | 0.5 |
| Composition containing polyurethane-59 | 2 |
| (PEG-240/decyltetradeceth-20/HDI) copolymer | 0.5 |
| Dipotassium glycyrrhizinate | 0.05 |
| Acetylated hyaluronic acid | 0.01 |
| Chelating agent | 0.05 |
| Buffer | q.s. |
| Terephthalylidene dicamphor sulfonic acid | 2 |
| Neutralizing agent | q.s. |
| Preservative | q.s. |
| PEG-12 dimethicone | 2 |
| Vitamin A derivative | 0.05 |
| Homosalate | 5 |
| Ethylhexyl salicylate | 5 |
| t-Butyl methoxydibenzoylmethane | 2 |
| Ethylhexyltriazine | 1 |
| Dioctyl succinate | 2 |
| Triethoxycaprylylsilane-treated fine particle zinc oxide | 7 |
| Hydrogen dimethicone-treated fine particle titanium oxide | 5 |
| Total | 100 |

### Formulation Example 2: Whitening Foundation

| | amount |
|---|---|
| Blended components | blended |
| Ion-exchange water | balance |
| Dipropylene glycol | 3 |
| Ethyl alcohol | 5 |
| Glycerin | 4 |
| PEG-40 hydrogenated castor oil | 1 |
| Succinoglycan | 0.2 |
| Composition containing polyurethane-59 | 3 |
| (PEG-240/decyltetradeceth-20/HDI) copolymer | 0.2 |
| Tranexamic acid | 2 |
| Chelating agent | 0.05 |
| Buffer | q.s. |
| Preservative | q.s. |
| PEG-12 dimethicone | 2 |
| Ethylhexyl methoxy cinnamate | 7.5 |
| Cetyl ethylhexanoate | 3.0 |
| Diisopropyl sebacate | 5.0 |
| Triethoxycaprylylsilane-treated pigment grade titanium oxide | 10 |
| Triethoxycaprylylsilane-treated iron oxide (red) | 0.4 |
| Triethoxycaprylylsilane-treated iron oxide (yellow) | 1 |
| Triethoxycaprylylsilane-treated iron oxide (black) | 0.01 |
| Starch powder | 3 |
| Total | 100 |

1. An oil-in-water emulsion cosmetic comprising:
(A) a hydrophobically modified polyether urethane having a specific structure comprising a urethane-based polymer obtained by reacting:
a monohydroxy compound (I) represented by the following general formula (1): 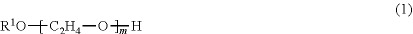
wherein R¹ is an aliphatic hydrocarbon group having 24 to 36 carbon atoms, and m represents a number of 0 to 1,000;
a polyethylene glycol (II) represented by the following general formula (2): 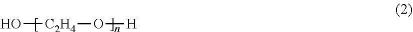
wherein n represents a number of 2 to 1,000;
a monoglyceryl ether compound (III) represented by the following general formula (3): 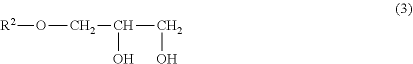
wherein R² represents an aliphatic hydrocarbon group having 5 to 12 carbon atoms; and
an isocyanate compound (IV) represented by the following general formula (4):
R³ [symbol] NCO]_{q} (4)
wherein R³ represents a hydrocarbon group having 4 to 13 carbon atoms, and q represents a number of 2 or 3;
(B) a polyether modified silicone having an HLB (Si) of 5 to 14; and
(C) a hydrophobized powder.

2. The cosmetic according to claim 1, further comprising (D) a lower alcohol and/or a polyol.

3. The cosmetic according to claim 1 or 2, further comprising (E) a nonionic surfactant having an HLB of 8 to 18.

4. The cosmetic according to any one of claims 1 or 2, wherein the (A) hydrophobically modified polyether urethane having a specific structure is polyurethane-59.

5. The cosmetic according to any one of claims 1 or 2, wherein the (C) hydrophobized powder is a metal oxide powder treated with triethoxycaprylylsilane, dimethicone, hydrogen dimethicone, or a metallic soap.

6. The cosmetic according to claim 3, wherein the (A) hydrophobically modified polyether urethane having a specific structure is polyurethane-59.

7. The cosmetic according to claim 3, wherein the (C) hydrophobized powder is a metal oxide powder treated with triethoxycaprylylsilane, dimethicone, hydrogen dimethicone, or a metallic soap.

8. The cosmetic according to claim 4, wherein the (C) hydrophobized powder is a metal oxide powder treated with triethoxycaprylylsilane, dimethicone, hydrogen dimethicone, or a metallic soap.

9. The cosmetic according to claim 6, wherein the (C) hydrophobized powder is a metal oxide powder treated with triethoxycaprylylsilane, dimethicone, hydrogen dimethicone, or a metallic soap.
